(19) United States
(12) Patent Application Publication
(10) Pub. No.: US 2026/0033776 A1
(43) Pub. Date: Feb. 5, 2026

(54) SYSTEM AND METHOD FOR DETECTING SLEEP APNEA

(71) Applicant: Northeastern University, Boston, MA (US)

(72) Inventors: Aarti Sathyanarayana, Brookline, MA (US); Shashank Manjunath, Providence, RI (US)

(21) Appl. No.: 19/118,274

(22) PCT Filed: Jan. 23, 2024
(86) PCT No.: PCT/US2024/012572

§ 371 Date: Apr. 3, 2025

Related U.S. Application Data
(60) Provisional application No. 63/481,123, filed on Jan. 23, 2023.

(51) Int. Cl. A61B 5/00; A61B 5/372; G16H 50/20

(52) U.S. Cl. CPC A61B 5/4818; A61B 5/372; G16H 50/20

(57) ABSTRACT

A computer-implemented method and corresponding computer-based system detect sleep apnea. The computer-implemented method transforms electroencephalogram (EEG) data into features. The EEG data is produced from EEG signals output over a window of time while a patient is awake. The EEG signals are output by at least two EEG electrodes coupled to the patient. The computer-implemented method further detects sleep apnea in the patient by applying a prediction model to the features. The features represent electrodynamics of a brain of the patient as measured over the window of time via the at least two EEG electrodes while the patient is awake. Such a computer-implemented method and computer-based system obviate an overnight sleep study of the patient in order to diagnose the patient as having sleep apnea.

### RELATED APPLICATION(S)

This application claims the benefit of U.S. Provisional Application No. 63/481,123, filed on Jan. 23, 2023. The entire teachings of the above application are incorporated herein by reference.

### BACKGROUND

Sleep apnea is a sleep disorder in which breathing repeatedly stops and starts. Risk factors for sleep apnea include age and obesity. Symptoms may include snoring loudly and feeling tired even after a full night's sleep. Treatment for sleep apnea often includes lifestyle changes, such as weight loss, and the use of a breathing assistance device at night, such as a continuous positive airway pressure (CPAP) machine.

### SUMMARY

According to an example embodiment, a computer-implemented method for detecting sleep apnea comprises transforming electroencephalogram (EEG) data into features. The EEG data is produced from EEG signals output over a window of time while a patient is awake. The EEG signals are output by at least two EEG electrodes coupled to the patient. The computer-implemented method further comprises detecting sleep apnea in the patient by applying a prediction model to the features. The features represent electrodynamics of a brain of the patient as measured over the window of time via the at least two EEG electrodes while the patient is awake.

The electrodynamics may represent neurons firing in the brain of the patient over the window of time while the patient is awake.

The prediction model may be a machine learning model. The applying may include employing, by the machine learning model, a multilayer perceptron (MLP), neural network, random forest, logistic regression method, or a combination thereof for non-limiting examples.

The transforming may include performing a topological data analysis (TDA) on the EEG data to determine a topology of coherence of the EEG signals. The features may further represent the topology of the coherence determined.

Transforming the EEG data into features may include performing a recurrence quantification analysis (RQA) on the EEG data to determine entropy of the EEG signals. The features may further represent the entropy determined.

The computer-implemented method may further comprise training the prediction model based on labels that discriminate whether the features are aligned with features of a control group of individuals that have not met at least one criterion for sleep apnea or a diagnostic group of individuals that have met the at least one criterion for sleep apnea.

Individuals of the control group and diagnostic group may be age matched, gender matched, or a combination thereof for non-limiting examples.

The window of time may be a multiple of thirty seconds for non-limiting example.

The EEG data may be clinical EEG data or consumer EEG data for non-limiting examples.

The sleep apnea detected may be obstructive sleep apnea for non-limiting example.

According to another example embodiment, a computer-based system for detecting sleep apnea comprises at least one memory and at least one processor. The at least one processor is configured to transform electroencephalogram (EEG) data into features. The EEG data is produced from EEG signals output over a window of time while a patient is awake. The EEG signals are output by at least two EEG electrodes coupled to the patient. The at least one processor is further configured to detect sleep apnea in the patient by applying a prediction model to the features. The features represent electrodynamics of a brain of the patient as measured over the window of time via the at least two EEG electrodes while the patient is awake.

Alternative computer-based system embodiments parallel those described above in connection with the example computer-implemented method embodiment.

According to yet another example embodiment, a non-transitory computer-readable medium for detecting sleep apnea has encoded thereon a sequence of instructions which, when loaded and executed by at least one processor, causes the at least one processor to transform electroencephalogram (EEG) data into features. The EEG data is produced from EEG signals output over a window of time while a patient is awake. The EEG signals are output by at least two EEG electrodes coupled to the patient. The sequence of instructions further causes the at least one processor to detect sleep apnea in the patient by applying a prediction model to the features. The features represent electrodynamics of a brain of the patient as measured over the window of time by the at least two EEG electrodes while the patient is awake.

Alternative non-transitory computer-readable medium embodiments parallel those described above in connection with the example computer-implemented method embodiment.

It should be understood that example embodiments disclosed herein can be implemented in the form of a method, apparatus, system, or non-transitory, computer-readable medium with program codes embodied thereon.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a block diagram of an example embodiment of a computer-based system for detecting sleep apnea.

FIG. 2 is a flow diagram of an example embodiment of a computer-implemented method for detecting sleep apnea.

FIG. 3 is a persistence diagram for an example embodiment of a delta band of an obstructive sleep apnea (OSA) positive patient.

FIG. 4 is a degree zero persistence landscape of an example embodiment of a delta band of a OSA positive patient.

FIG. 5 is a block diagram of an example internal structure of a computer optionally within an embodiment disclosed herein.

### DETAILED DESCRIPTION

A description of example embodiments follows.

While sleep apnea may be described herein with regard to a pediatrics, it should be understood that an example embodiment for detection of sleep apnea disclosed herein is not limited to pediatrics.

### I. Overview

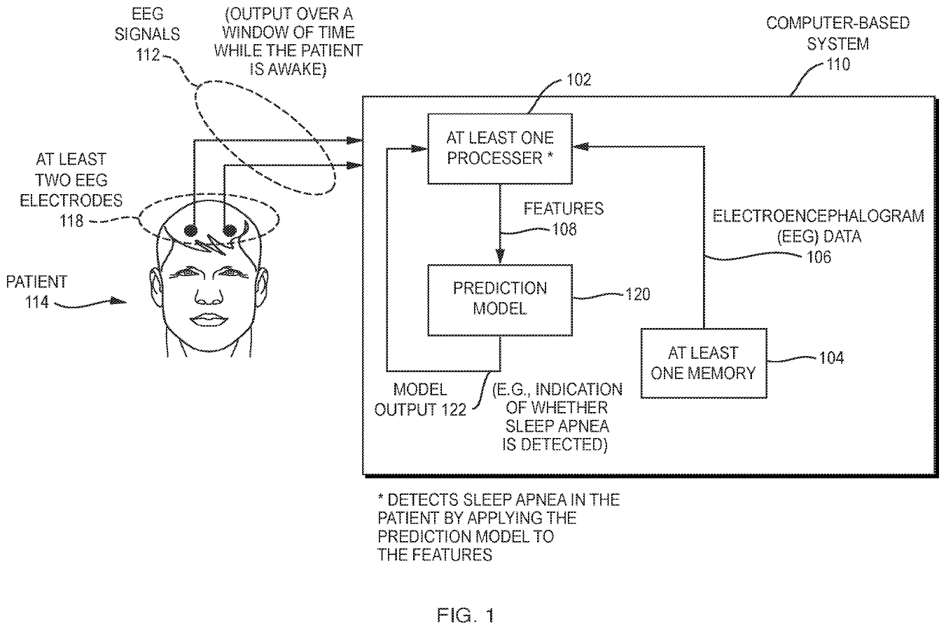

Pediatric Obstructive Sleep Apnea (OSA) is difficult to diagnose and leads to significant complications, including poor rest and heart disease (Oscar Sans Capdevila, et al., “Pediatric Obstructive Sleep Apnea. Proceedings of the American Thoracic Society;” 5(2): 274-282, February 2008). In children especially, OSA can lead to behavioral changes and further reduction in quality of life. Identification of OSA in patients typically requires an overnight polysomnogram (PSG) (Michael S. Schechter and Section on Pediatric Pulmonology, Sub-committee on Obstructive Sleep Apnea Syndrome. Technical report: Diagnosis and management of childhood obstructive sleep apnea syndrome. Pediatrics, 109(4): e69, April 2002). Polysomnograms include a number of sensors, including electroencephalogram (EEG) data, pulse oximetry, and respiratory rate, among others. Acquisition of this information is difficult for the patient, as they have to sleep in an unknown environment while being observed by medical personnel, as well as sleep with multiple sensors connected to their body. Polysomnograms are considered the gold standard for OSA diagnosis and attempts to use other clinical methods, such as questionnaires, lead to poor diagnostic accuracy (Michael S. Schechter and Section on Pediatric Pulmonology, Sub-committee on Obstructive Sleep Apnea Syndrome. Technical report: Diagnosis and management of childhood obstructive sleep apnea syndrome. Pediatrics, 109(4): e69, April 2002). An example embodiment disclosed herein enables sleep apnea to be detected from “awake” electroencephalogram (EEG) data, that is, EEG data that is captured via EEG electrodes coupled to a patient while the patient is awake. Such an example embodiment obviates existing diagnostic methods that are based on either an inpatient overnight study (very expensive and a bottleneck for patients to schedule the study) or an outpatient overnight study (not as expensive but also a bottleneck). An example embodiment of a computer-based system that detects sleep apnea form such awake EEG data is disclosed below with regard to FIG. 1.

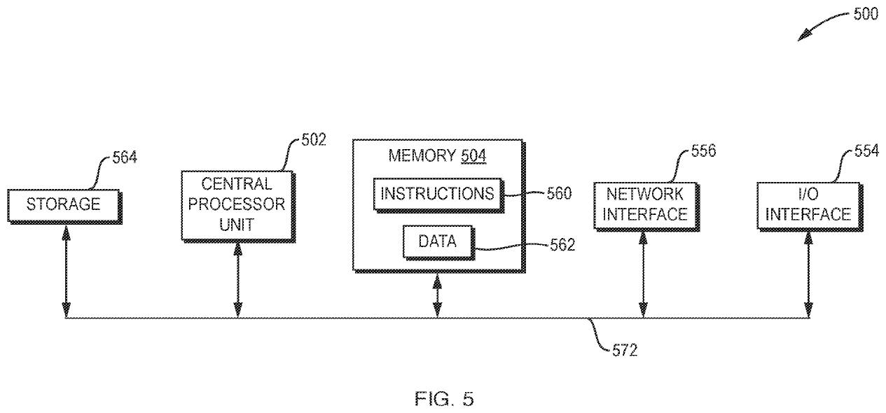

FIG. 1 is a block diagram of an example embodiment of a computer-based system 110 for detecting sleep apnea. The computer-based system 110 comprises at least one processor 102 and at least one memory 104, such as the central processor unit (CPU) 502 and memory 504 disclosed further below with regard to FIG. 5 for non-limiting example. Continuing with reference to FIG. 1, the at least one processor 102 may be configured to transform electroencephalogram (EEG) data 106 into features 108. Such EEG data 106 may be stored in the at least one memory 104 for non-limiting example. The EEG data 106 may be produced from EEG signals 112 output over a window of time (not shown) while a patient 114 is awake. The EEG signals 112 may be output by at least two EEG electrodes 118 that are coupled to the patient 114. The at least one processor 102 may be further configured to detect sleep apnea in the patient 114 by applying a prediction model 120 to the features 108. For non-limiting example, the predication model 120 may output model output 122 that represents an indication, such a likelihood for non-limiting example, of whether sleep apnea has been detected. The features 108 may represent electrodynamics (not shown) of a brain (not shown) of the patient 114 as measured over the window of time via the at least two EEG electrodes 118 while the patient 114 is awake.

The window of time may be a multiple of thirty seconds for non-limiting example. As such, according to an example embodiment, sleep apnea may be detected based on thirty seconds of awake EEG data obviating the patient 114 from being subjected to an overnight sleep study. The electrodynamics may represent neurons (not shown) firing in the brain (not shown) of the patient 114 over the window of time while the patient 114 is awake. The prediction model 120 may be a machine learning model and the applying may include employing, by the machine learning model, a multilayer perceptron (MLP), neural network, random forest, logistic regression method, or a combination thereof for non-limiting examples.

The at least one processor 102 may be further configured to perform a topological data analysis (TDA) (not shown) on the EEG data 106 to determine a topology of coherence (not shown) of the EEG signals 112. The features 108 may further represent the topology of the coherence determined for non-limiting example. TDA is an emerging signal processing technique for biological signal processing. TDA leverages the invariant topological features of signals in a metric space in order to help understand the space. This technique allows for robust analysis of biological signals, even in the presence of noise. As disclosed further below, TDA may be applied to brain connectivity networks derived from EEG signals to identify statistical differences between pediatric patients with OSA and pediatric patients without OSA for non-limiting example. A large corpus of data was leveraged, as disclosed further below, and shows that TDA enables a statistical difference between the two groups. Such disclosure establishes that TDA may be used as a tool to identify obstructive sleep apnea without use of a full polysomnogram study.

Continuing with reference to FIG. 1, the at least one processor 102 may be further configured to perform a recurrence quantification analysis (RQA) (not shown) on the EEG data 106 to determine entropy (not shown) of the EEG signals 112. The features 108 may further represent the entropy determined for non-limiting example.

The at least one processor 102 may be further configured to train the prediction model 120 based on labels (not shown) that discriminate whether the features 108 are aligned with features of a control group of individuals (not shown) that have not met at least one criterion (not shown) for sleep apnea or a diagnostic group of individuals (not shown) that have met the at least one criterion for sleep apnea. Individuals of the control group and diagnostic group may be age matched, gender matched, or a combination thereof for non-limiting examples.

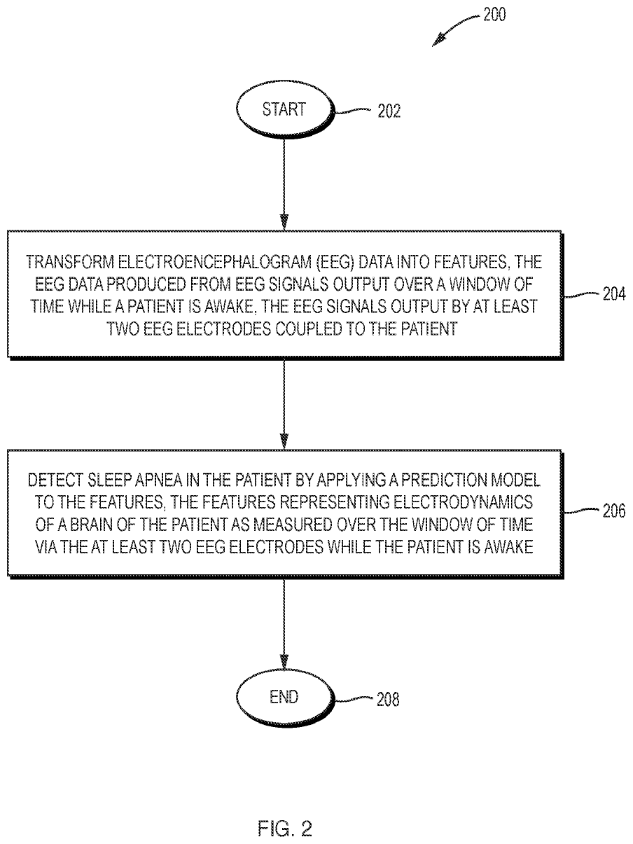

The EEG data 106 may be clinical EEG data or consumer EEG data for non-limiting examples. The sleep apnea detected may be obstructive sleep apnea (OSA) for non-limiting example. Such detection of sleep apnea may be performed via a computer-implemented method, such as the computer-implemented method disclosed below with regard to FIG. 2.

FIG. 2 is a flow diagram 200 of an example embodiment of a computer-implemented method for detecting sleep apnea. The method begins (202) and comprises transforming electroencephalogram (EEG) data into features, the EEG data produced from EEG signals output over a window of time while a patient is awake, the EEG signals output by at least two EEG electrodes coupled to the patient (202). The computer-implemented method further comprises detecting sleep apnea in the patient by applying a prediction model to the features, the features representing electrodynamics of a brain of the patient as measured over the window of time via the at least two EEG electrodes while the patient is awake (204). The computer-implemented method thereafter ends (206) in the example embodiment. Further technical details are disclosed below.

An example embodiment disclosed herein may employ topological data analysis (TDA) to analyze EEG signals to assist with identification of OSA before disordered breathing occurs. Topological data analysis leverages ideas from the mathematical field of topology and applies these ideas to the analysis of concrete signals (Frédéric Chazal and Bertrand Michel, “An Introduction to Topological Data Analysis: Fundamental and Practical Aspects for Data Scientists;” Frontiers in Artificial Intelligence, 4, 2021). Broadly, TDA enables an exploitation of the topological and geometric structures inherent to data and an example embodiment disclosed herein may use these structures to study fundamental differences between the EEG signals of OSA positive and OSA negative individuals.

### II

### A. Obstructive Sleep Apnea

Snoring is relatively common in children, with about 10% incidence in children ages 1-9. However, obstructive sleep apnea only occurs in 2-3% of children with habitual snoring (Oscar Sans Capdevila, et al., “Pediatric Obstructive Sleep Apnea. Proceedings of the American Thoracic Society;” 5(2): 274-282, February 2008). Further complication is caused by recent increases in pediatric obesity. Pediatric OSA is typically caused by adenotonsillary hypertrophy. Pediatric OSA has been correlated with poor growth and failure to thrive (James Chan, et al., “Obstructive Sleep Apnea in Children;” 69(5), 2004). Furthermore, daytime drowsiness (somnolescence) is much less common in children with OSA as compared to adults with OSA, leading to further difficulties with the diagnosis of OSA.

### B. Topological Data Analysis

Topological data analysis aims to identify quantitative information about the structure of data and leverage this information for downstream data analysis tasks (Frédéric Chazal and Bertrand Michel, “An Introduction to Topological Data Analysis: Fundamental and Practical Aspects for Data Scientists;” Frontiers in Artificial Intelligence, 4, 2021). Data is assumed to exist in a metric space, allowing distances between different pieces of data to be computed, which are typically viewed as points in an ambient space. Once such data is computed, an attempt to build a shape on a point cloud may be made by varying the distance with which two points are considered as connected (further discussed in Section III-C).

The useful assumption in the work disclosed herein is that the brain connectivity network for OSA positive patients has a fundamentally different topological structure than the brain connectivity network for OSA negative patients. In order to identify if the brain connectivity networks are fundamentally topologically different, it is useful to understand how to differentiate topological spaces. In particular, one can define two invariants between topological spaces: Simplicial homology groups, and Betti numbers. The definition of a simplicial complex is provided below.

Definition II.1 (Simplicial Complex (Herbert Edelsbrunner and John Harer, “Computational Topology: An Introduction.”). A simplicial complex is a finite collection of simplices K such that σ∈K and τ≤σ implies that τ∈K, and σ, σ0∈K implies σ∪σ0 is either empty or a face of both.

If there are two topological spaces defined by simplicial complexes which are homotopy equivalent (as in Section III-C), then their homology groups are isomorphic and they have the same Betti numbers.

Definition II.2 (Simplicial Homology Group (Frédéric Chazal and Bertrand Michel, “An Introduction to Topological Data Analysis: Fundamental and Practical Aspects for Data Scientists;” Frontiers in Artificial Intelligence, 4, 2021)). The kth (simplicial) homology group of a finite simplicial complex K is the quotient vector space:

$${H}_{k} ( K ) = {Z}_{k} ( K ) / {B}_{k} ( K ) ,$$

where Zₖ(K) is the kernel of K and Bk (K) is the boundary of K.

Definition II.3 (Betti Number (Frédéric Chazal and Bertrand Michel, “An Introduction to Topological Data Analysis: Fundamental and Practical Aspects for Data Scientists;” Frontiers in Artificial Intelligence, 4, 2021)). The kth Betti number of a finite simplicial complex K is the dimension βₖ(K)=dim Hₖ(K) of the vector space Hₖ(K).

In this work, an attempt to understand the topological features of each homology group (e.g., Betti number) for the brain connectivity network of OSA positive and OSA negative patients is disclosed.

### III. Methods

### A. Data

In this work, the Nationwide Children's Hospital (NCH) Sleep DataBank (SDB) (Guo-Qiang Zhang, et al., “The National Sleep Research Resource: Towards a sleep data commons;” Journal of the American Medical Informatics Association: JAMIA, 25(10): 1351-1358 October 2018), (Harlin Lee, et al., “A large collection of real-world pediatric sleep studies;” Scientific Data, 9(1): 421, July 2022) is leveraged. This dataset includes 3,984 pediatric patients. This dataset contains a significant volume of data from a number of sensors, including EEG, electromyogram, electrooculogram, electrocardiogram, nasal and oral sensors to measure airflow, and pulse oximetry, among others. This large volume of data and sensors allows us to study pediatric OSA across a wide variety of pediatric patients. The dataset additionally contains annotations of sleep events, such as N1 sleep, N2 sleep, N3 sleep, REM sleep, and awake state, in 30 second intervals. We focus on EEG signals collected at 256 Hz using 7-channel EEG studies, which restricts us to sleep studies from 2883 patients.

### B. Signal Processing

First, s basic signal processing is performed on EEG signals. Filtering out 60 Hz and 120 Hz power noise using a 3rd order Butterworth bandstop filter is performed.

Techniques from Bourakna et al. may be leveraged to create distance matrices from our EEG data (Anass El Yaagoubi Bourakna, et al., “Topological Data Analysis for Multivariate Time Series Data;” April 202). Data is processed on a per-study basis. First, all available channels are extracted from a PSG study, leading to a set of time-series signals Xₜ(t) for channel i∈V and time t∈{1, · · · 7}. A smoothed periodogram is then created from each channel using the following equations:

$$d ( {ω}_{k} ) = \frac{1}{√ T} {\sum }_{t = 1}^{T} {X}_{i} ( t ) exp ( - i 2 π t {ω}_{k} ) \qquad ( 1 )$$

$$\hat{f} ( {ω}_{k} ) = {\sum }_{ω} {k}_{h} ( ω - {ω}_{k} ) d ( {ω}_{k} ) d ( {ω}_{k} ) * \qquad ( 2 )$$

where kₕ(ω-ωk) indicates a smoothing kernel centered around ωₖ and h is a bandwidth parameter. From these smoothed periodograms, the cross-correlation between EEG channels i and j may be calculated:

$$C ( {X}_{i} , {X}_{j} , ω ) = \frac{{\vert {f}_{i , j , ( ω )} \vert}^{2}}{{f}_{i , i ( ω )} {f}_{j , j ( ω )}} \qquad ( 3 )$$

Then, a decreasing function may be leveraged to create a distance between coherence values for a given frequency ω:

$$( {X}_{i} , {X}_{j} , ω ) = 1 - C ( {X}_{i} , {X}_{j} , ω ) \qquad ( 4 )$$

This distance matrix serves as the foundation for TDA methods disclosed herein.

### C. Topological Data Analysis Methods

In this analysis persistent homology techniques on the distance matrices described in Section III-B are leveraged which, in turn, describe the distances between points in a point cloud. While a number of topological approaches to data exist, an example embodiment may focus on persistent homology techniques. Once a distance matrix for a 30-second signal is obtained, an abstract simplicial complex may be built on the data, in particular a Vietoris-Rips Complex, or Rips complex, and furthermore the Rips filtration using the Python package ripserplusplus (Simon Zhang, et al., “GPU-Accelerated Computation of Vietoris-Rips Persistence Barcodes:” October 2020).

Since a distance matrix of the data is obtained, it can be said that the data exists in a metric space. It is useful to understand the underlying shape of the point cloud based on the distance matrix. Let S be our points in [symbol] ⁿ described by the previous distance matrix D. The Rips complex R of the data S and distance r is given by the abstract simplicial complex consisting of all subsets of diameter at most r, i.e.

$$( S , r ) = \{ σ ⊂ S \vert diam ( σ ) \leq r \}$$

Note that R is inherently tied to a given r value. It is useful to understand how the Rips complexes vary as r is varied. Therefore, a filtration is constructed, called a Rips filtration. A Rips filtration is the set of Rips complexes created by varying the free parameter r. An example embodiment may start with a small r (say, r=0), and increase r continuously to obtain a set of subcomplexes of the simplicial complex (Herbert EdelsbrunS^{r}Sᵣᵣner and John Harer. “Computational Topology: An Introduction.”):

$$Ø = {K}_{0} ⊆ {K}_{1} ⊆ {K}_{2} ⊆ ⊆ {K}_{m}$$

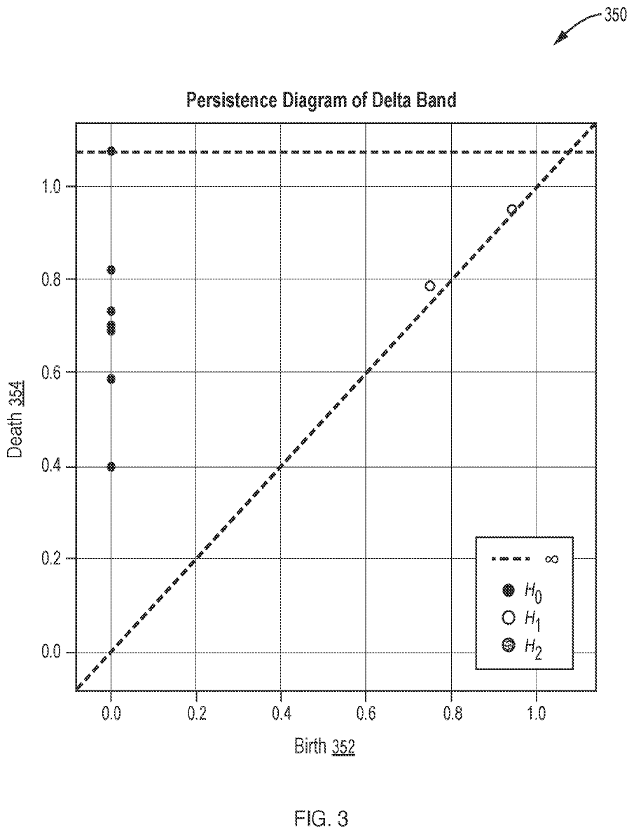

An example embodiment may leverage ripserplusplus to compute the Rips filtration. Once this set of simplicial complexes has been computed, it is useful to understand how the topological features of the data vary as r is varied. Asr is varied from Kᵢ−1 to Kᵢ, the topological features change, and there is a gain or loss of topological features. An example embodiment may quantify the r at which a particular topological feature in a particular homology group appears and the r at which the same topological feature disappears as a (birth, death) pair, which exists in [symbol] ². These points can be plotted on a graph to create a persistence diagram, such as the persistence diagram of FIG. 3, disclosed below.

FIG. 3 is a persistence diagram 350 for an example embodiment of a delta band of a OSA positive patient. In the persistence diagram 350, points which are closer to the diagonal (i.e., which have a birth r 352 that is close to the death r 354) are considered to be spurious features: points which are far off the diagonal (i.e. have a birth r that is significantly smaller than the death r) are considered to be significant features (Jose A. Perea, “Topological Time Series Analysis;” November 2018).

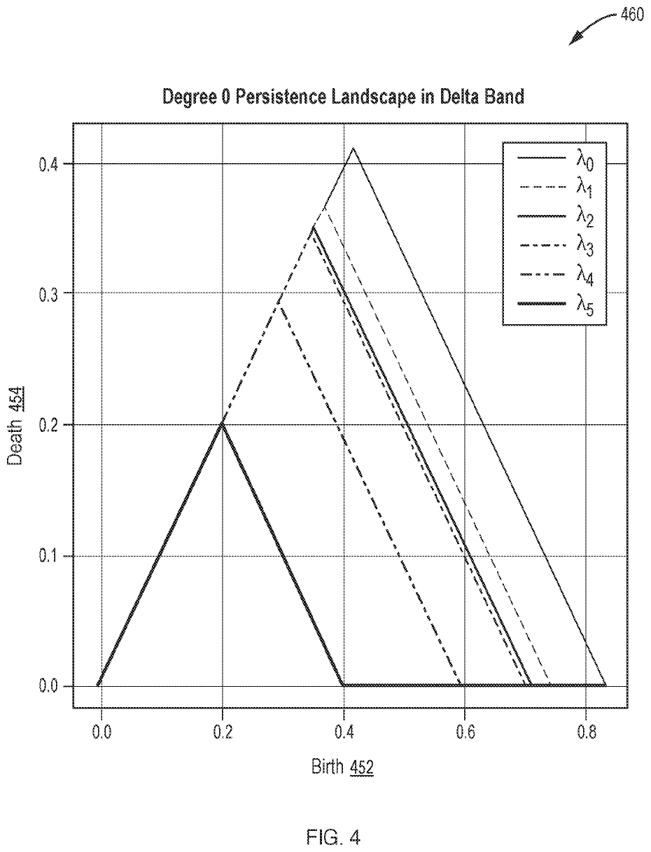

While the persistence diagram 350 is an easy way to visualize the topological features of a particular sample, it is difficult to leverage for further statistical analysis. This is because persistence diagrams are not functions, and do not have any vector space structure. Persistence diagrams therefore do not extend to separable Banach spaces, and therefore do not lend themselves to analysis through the lens of random variables (Peter Bubenik, “Statistical Topological Data Analysis using Persistence Landscapes”). However, one can easily transform the persistence diagram 350 into a persistence landscape, which is a function and does exist in a separable Banach space, such as the persistence landscape of FIG. 4, disclosed below.

FIG. 4 is a degree 0 persistence landscape 460 of an example embodiment of a delta band of a OSA positive patient. The persistence landscape 460 may be constructed by drawing an isosceles triangle centered on the points (birth 452, death 454) of the persistence diagrams. When intersections occur the highest function is defined as the persistence landscape (Peter Bubenik, “Statistical Topological Data Analysis using Persistence Landscapes”).

### D. Permutation Testing

While a usable TDA feature in the persistence landscapes has been obtained, it is useful to identify a test which can indicate whether the TDA features from OSA positive pediatric patients is different than the TDA features from OSA negative patients. To this end, a permutation test was leveraged, as described in (Andrew Robinson and Katharine Turner, “Hypothesis Testing for Topological Data Analysis;” February 2016). In this methodology, a null hypothesis H₀ is that there is no impact of OSA on the brain connectivity network. To perform the test, the following workflow was implemented:
1) Calculate the average OSA positive persistence landscapes

$$\{ {λ}_{1}^{( 1 )} , … , {λ}_{n 1}^{( 1 )} \}$$

and the average OSA negative persistence landscapes

$$\{ {λ}_{1}^{( 2 )} , … , {λ}_{n 2}^{( 2 )} \} .$$

2) Calculate the difference between the average OSA positive persistence landscape and the average OSA negative persistence landscape and find the most significant value of this difference landscape \hat{F}_{T}.
3) Permute the labels of the two groups such that mi randomly chosen positively labeled persistence landscapes are chosen and the remaining m landscapes are negatively labeled.
4) Calculate the difference between the permuted positive group and the permuted negative group, and find the most significant value of this difference landscapes \hat{F}ₚ
5) If \hat{F}ₚ≥\hat{F}_{T}, count a significant permutation.
6) Repeat steps 3 and 4 K times.
7) Compute the p-value as the number of significant permutations S divided by the total number of per-mutations K

### IV. Results

The p-values for the TDA permutation tests discussed in Section III-D are presented in the tables below, in which p-values with p<0.05 are considered to be significant.

TABLE 1
p-VALUES FOR SLEEP STATES USING TDA

| EEG Band | N1 Sleep | N2 Sleep | N3 Sleep | REM Sleep |
|---|---|---|---|---|
| Delta Band | 0.000 | 0.001 | 0.000 | 0.040 |
| Theta Band | 0.000 | 0.000 | 0.000 | 0.000 |
| Alpha Band | 0.000 | 0.000 | 0.000 | 0.000 |
| Beta Band | 0.000 | 0.000 | 0.000 | 0.000 |
| Gamma Band | 0.000 | 0.000 | 0.000 | 0.000 |

For sleep states, it was that TDA can identify differences between OSA positive and OSA negative cohorts for all sleep states and all frequency bands.

Now presented are the p-value results for awake states taken during PSG studies. “Awake before” indicates signals taken when the patient is awake before their first sleep state, “Awake During” indicates signals taken when the patient is awake in the middle of the sleep study, and “Awake After” includes signals taken once the patient has woken up at the end of the study but before the study is completed.

TABLE II
p-VALUES FOR AWAKE STATES USING TDA

| EEG Band | Awake Before | Awake During | Awake After |
|---|---|---|---|
| Delta Band | 0.002 | 0.000 | 0.863 |
| Theta Band | 0.001 | 0.000 | 0.940 |
| Alpha Band | 0.010 | 0.004 | 0.899 |
| Beta Band | 0.000 | 0.000 | 0.252 |
| Gamma Band | 0.029 | 0.055 | 0.312 |

These results indicate that an example embodiment disclosed herein can identify statistically significant differences for all awake before frequency bands, the delta, theta, alpha, and beta bands in the awake during study case, and none of the frequency bands in the awake after study case.

Thus, as disclosed herein, the brain dependence networks of OSA positive pediatric patients show a statistically significant difference than the brain dependance networks of OSA negative pediatric patients when analyzed using topological data analysis. While this analysis is useful on large cohorts of patients, it is not useful for EEG scans taken on single patients. Further work includes developing techniques to directly analyze single patient EEG scans for OSA.

NCH Sleep DataBank was supported by the National Institute of Biomedical Imaging and Bioengineering of the National Institutes of Health under Award Number R01EB025018. The National Sleep Research Resource was supported by the U.S. National Institutes of Health, National Heart Lung and Blood Institute (R24 HL114473, 75N92019R002).

FIG. 5 is a block diagram of an example of the internal structure of a computer 500 in which various embodiments of the present disclosure may be implemented. The computer 500 contains a system bus 552, where a bus is a set of hardware lines used for data transfer among the components of a computer or digital processing system. The system bus 552 is essentially a shared conduit that connects different elements of a computer system (e.g., processor, disk storage, memory, input/output ports, network ports, etc.) that enables the transfer of information between the elements. Coupled to the system bus 552 is an I/O device interface 554 for connecting various input and output devices (e.g., keyboard, mouse, display monitors, printers, speakers, etc.) to the computer 500. A network interface 556 allows the computer 500 to connect to various other devices attached to a network (e.g., global computer network, wide area network, local area network, etc.). Memory 504 provides volatile or non-volatile storage for computer software instructions 560 and data 562 that may be used to implement embodiments (e.g., method of the flow diagram 200) of the present disclosure, where the volatile and non-volatile memories are examples of non-transitory media. Disk storage 564 provides non-volatile storage for computer software instructions 560 and data 562 that may be used to implement embodiments (e.g., method of the flow diagram 200) of the present disclosure. A central processor unit (CPU) 502 is also coupled to the system bus 552 and provides for the execution of computer instructions.

Example embodiments disclosed herein may be configured using a computer program product; for example, controls may be programmed in software for implementing example embodiments. Further example embodiments may include a non-transitory, computer-readable medium containing instructions that may be executed by a processor, and, when loaded and executed, cause the processor to complete methods described herein. It should be understood that elements of the block and flow diagrams may be implemented in software or hardware, such as via one or more arrangements of circuitry of FIG. 5, disclosed above, or equivalents thereof, firmware, a combination thereof, or other similar implementation determined in the future.

In addition, the elements of the block and flow diagrams described herein may be combined or divided in any manner in software, hardware, or firmware. If implemented in software, the software may be written in any language that can support the example embodiments disclosed herein. The software may be stored in any form of computer readable medium, such as random-access memory (RAM), read-only memory (ROM), compact disk read-only memory (CD-ROM), and so forth. In operation, a general purpose or application-specific processor or processing core loads and executes software in a manner well understood in the art. It should be understood further that the block and flow diagrams may include more or fewer elements, be arranged or oriented differently, or be represented differently. It should be understood that implementation may dictate the block, flow, and/or network diagrams and the number of block and flow diagrams illustrating the execution of embodiments disclosed herein.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A computer-implemented method for detecting sleep apnea, the computer-implemented method comprising:
transforming electroencephalogram (EEG) data into features, the EEG data produced from EEG signals output over a window of time while a patient is awake, the EEG signals output by at least two EEG electrodes coupled to the patient; and
detecting sleep apnea in the patient by applying a prediction model to the features, the features representing electrodynamics of a brain of the patient as measured over the window of time via the at least two EEG electrodes while the patient is awake.

2. The computer-implemented method of claim 1, wherein the electrodynamics represent neurons firing in the brain of the patient over the window of time while the patient is awake.

3. The computer-implemented method of claim 1, wherein the prediction model is a machine learning model and wherein the applying includes employing, by the machine learning model, a multilayer perceptron (MLP), neural network, random forest, logistic regression method, or a combination thereof.

4. The computer-implemented method of claim 1, wherein the transforming includes performing a topological data analysis (TDA) on the EEG data to determine a topology of coherence of the EEG signals and wherein the features further represent the topology of the coherence determined.

5. The computer-implemented method of claim 1, wherein transforming the EEG data into features includes performing a recurrence quantification analysis (RQA) on the EEG data to determine entropy of the EEG signals and wherein the features further represent the entropy determined.

6. The computer-implemented method of claim 1, further comprising training the prediction model based on labels that discriminate whether the features are aligned with features of a control group of individuals that have not met at least one criterion for sleep apnea or a diagnostic group of individuals that have met the at least one criterion for sleep apnea.

7. The computer-implemented method of claim 6, wherein individuals of the control group and diagnostic group are age matched, gender matched, or a combination thereof.

8. The computer-implemented method of claim 1, wherein the window of time is a multiple of thirty seconds.

9. The computer-implemented method of claim 1, wherein the EEG data is clinical EEG data or consumer EEG data.

10. The computer-implemented method of claim 1, wherein the sleep apnea detected is obstructive sleep apnea.

11. A computer-based system for detecting sleep apnea, the computer-based system comprising:
at least one memory; and
at least one processor configured to transform electroencephalogram (EEG) data into features, the EEG data produced from EEG signals output over a window of time while a patient is awake, the EEG signals output by at least two EEG electrodes coupled to the patient, the at least one processor further configured to detect sleep apnea in the patient by applying a prediction model to the features, the features representing electrodynamics of a brain of the patient as measured over the window of time via the at least two EEG electrodes while the patient is awake.

12. The computer-based system of claim 11, wherein the electrodynamics represent neurons firing in the brain of the patient over the window of time while the patient is awake.

13. The computer-based system of claim 11, wherein the prediction model is a machine learning model and wherein the applying includes employing, by the machine learning model, a multilayer perceptron (MLP), neural network, random forest, logistic regression method, or a combination thereof.

14. The computer-based system of claim 11, wherein the at least one processor is further configured to perform a topological data analysis (TDA) on the EEG data to determine a topology of coherence of the EEG signals and wherein the features further represent the topology of the coherence determined.

15. The computer-based system of claim 11, wherein the at least one processor is further configured to perform a recurrence quantification analysis (RQA) on the EEG data to determine entropy of the EEG signals and wherein the features further represent the entropy determined.

16. The computer-based system of claim 11, wherein the at least one processor is further configured to train the prediction model based on labels that discriminate whether the features are aligned with features of a control group of individuals that have not met at least one criterion for sleep apnea or a diagnostic group of individuals that have met the at least one criterion for sleep apnea.

17. The computer-based system of claim 16, wherein individuals of the control group and diagnostic group are age matched, gender matched, or a combination thereof.

18. The computer-based system of claim 11, wherein the window of time is a multiple of thirty seconds.

19. The computer-based system of claim 11, wherein the EEG data is clinical EEG data or consumer EEG data and wherein the sleep apnea detected is obstructive sleep apnea.

20. A non-transitory computer-readable medium for detecting sleep apnea, the non-transitory computer-readable medium having encoded thereon a sequence of instructions which, when loaded and executed by at least one processor, causes the at least one processor to:
transform electroencephalogram (EEG) data into features, the EEG data produced from EEG signals output over a window of time while a patient is awake, the EEG signals output by at least two EEG electrodes coupled to the patient; and
detect sleep apnea in the patient by applying a prediction model to the features, the features representing electrodynamics of a brain of the patient as measured over the window of time by the at least two EEG electrodes while the patient is awake.
